(19) United States
(12) Patent Application Publication
(10) Pub. No.: US 2006/0141445 A1
(43) Pub. Date: Jun. 29, 2006

(54) Method for manufacturing a sensor chip and sensor chip blank

(75) Inventor: Holger Klapproth, Freiburg (DE)

Correspondence Address: THE WEBB LAW FIRM, P.C., 700 KOPPERS BUILDING, 436 SEVENTH AVENUE, PITTSBURGH, PA, 15219, US

(73) Assignee: Micronas Holding GmbH, Freiburg i. Br. (DE)

(21) Appl. No.: 11/316,674

(22) Filed: Dec. 22, 2005

(30) Foreign Application Priority Data
Dec. 23, 2004 (EP) 04 030 547.6

(51) Int. Cl. C12Q 1/00

(52) U.S. Cl. 435/4

(57) ABSTRACT

In a process for manufacturing a sensor chip a support is prepared having at least one surface region in which at least one reactive group is arranged. A buffer solution is applied to the surface region for coating at least one surface region. Then, at least one receptor having binding specificity for a ligand to be detected is brought into contact with the surface coating so obtained in such a manner that said receptor binds covalently to at least one reactive group.

The invention relates to a method for manufacturing a sensor chip wherein a support is prepared that has at least one surface region in which at least one reactive group is arranged. The invention further relates to a sensor chip blank that has a support that has at least one surface region in which at least one reactive group is arranged, which is suitable for covalently binding to a receptor.

This type of method for manufacturing a sensor chip operating on the receptor-ligand principle is well known from DE 102 33 303 A1. Here receptors are covalently immobilized on a surface region of a support. The receptors are first dissolved in a buffer solution and then the buffer solution is applied to the surface region. Using a buffer solution, the pH changes substantially less strongly when an acid or base is added than would be the case if an unbuffered solution were to be used. Application of the buffer solution containing the receptors is done preferably with the aid of a printer such as a matrix printer and/or a piezo stack printer.

The binding of the receptors is specific for a ligand that is to be detected and that is presumed to be contained in an analyte being tested. In order to detect the ligand the analyte is initially brought into contact for a period of time with the receptor that has previously been immobilized on the support, said time being sufficient for a representative quantity of any ligands contained in the analyte to bind to the receptors. Then it is examined as to whether binding has occurred. In addition, the surface region of the support can, for example, be irradiated with an excitation radiation that induces a luminescence as a factor of the binding of the ligand to the receptor and which is measured using an optical radiation receiver. A receptor may be nucleic acids or derivatives thereof (DNA, RNA, PNA, LNA, oligonucleotides, plasmids, chromosomes), peptides, proteins (enzyme, protein, oligopeptides, cellular receptor proteins and their complexes, peptide hormones, antibodies and their fragments), carbohydrates and their derivatives, in particular glycosylated proteins and glycosides, fats, fatty acids and/or lipids.

Covalent binding of the receptor to the surface region is dependent on the pH and the buffering capacity of the buffer solution. Nevertheless, all desired buffer solutions cannot be reliably printed. Thus buffer solutions, but particularly those having a relatively high salt concentration due to their pH, are problematic because they tend to crystallize very rapidly. The capillary or nozzle of the print pin can clog as a result of the crystals. It is also possible for the buffer solution to crystallize even before it reaches the printer such as in a receptacle depression or a microtiter plate, for example, in which the buffer solution is stored. Another drawback is the fact that sensitive receptors must often be cooled in order to remain usable. However, the risk of crystallization is further increased by virtue of the cooling.

A specific pH is required in order to obtain effective covalent binding of the receptors to the support and that pH is made possible by the buffer capacity. At high receptor concentrations, consequently high salt concentrations are also required in the buffer solution. However, in practice these cannot be realized offhand if the buffer solution can no longer be printed. A further drawback of the method resides in the fact that the receptors must be soluble in the buffer solution so that they can be printed on the surface region of the support.

Therefore, there is the problem of providing a method of the aforementioned type that makes it possible for the receptors to bind effectively to the surface region of the support, yet wherein said method allows the receptors to be applied in simple fashion to the surface region of the support such as by using a printer having a capillary system, for example. Furthermore, there is the problem of providing a sensor chip blank of the aforementioned type that is suitable for use in the method.

This problem is solved with regard to the method such that a support is prepared, which has at least one surface region having at least one reactive group and in that a buffer solution is applied to said surface region for coating the at least one surface region and subsequently at least one receptor having binding specificity for a ligand to be detected is brought into contact with the said surface coating so obtained, such that it binds to at least one reactive group.

Thus initially the buffer solution is applied without the receptors on the support and only after this is done is the receptor brought into contact with the support's surface coating obtained by applying the buffer solution. Here, the receptor is preferably applied in solution to the surface coating, whereby the solvent, in which the receptor is dissolved, can be differentiated from the buffer solution. This makes it advantageously possible to select a solvent that makes it possible to easily apply the receptor without the risk that solids crystallize, precipitated and/or separate out of the solution prior to or during application of the receptor. However, a high concentration of the buffer solution or a buffer substance contained therein can be attained in the surface region of the support so that the receptor can bind covalently to at least one reactive group arranged in the surface region of the support. The covalent binding can occur by way of radical processes and/or nucleophilic additions.

In an advantageous embodiment of the invention, a solvent present in the buffer solution can be eliminated, preferably by evaporation, from the surface region so that a buffer substance contained in the buffer solution remains as a solid, paste-like and/or gel-like coating region on the support and so that subsequently the receptor is applied to this coating region. The sensor chip blank obtained by application of the buffer solution and on which no receptors have yet been immobilized can be easily handled and can also be stored exceptionally well until needed. Especially high buffer substance concentrations can be obtained in the surface region by removing the solvent so that the receptor can be applied in correspondingly high concentrations on the support and immobilized thereon.

It is preferable that such a buffer solution be applied to at least one surface region of the support so that a plurality of buffer solution volumes are formed arranged laterally separated from each other, preferably in a matrix in a plurality of rows or columns. Accordingly, tests can be done on the support at several sites at the same time and/or at different times.

It is advantageous if the buffer solution is applied to at least one surface region of the support using a printing process, in particular a pad printing process. Here it is even possible to print several or all buffer volumes of the matrix onto the support simultaneously. The method according to the invention can thus be implemented quickly and inexpensively. The buffer solution can be applied at a higher temperature to the at least one surface region of the support than the temperature of application of the at least one receptor. Accordingly, a longer shelf life is ensured in the case of heat-sensitive receptors. Furthermore, the risk of the buffer solution crystallizing before it reaches the support is reduced.

It is particularly advantageous if at least one receptor is dissolved in a solvent and then, using a jet printer, printed or sprayed onto the surface region. In this fashion, the method can be used even more cost-effectively. In order to ensure that the receptor can be precisely positioned on the surface coating previously produced on the support by application of the buffer solution, an optical sensor for detecting the position of the surface coating relative to an outlet opening or nozzle of the jet printer can be provided. The sensor can be combined with a positioning device that positions the outlet opening as a factor of a sensor-measurement signal so that the receptor is placed directly on the surface coating.

Concerning the aforementioned sensor chip blank, the aforementioned problem is solved in that at least one solid, paste-like and/or gel-like coating area containing at least one buffer substance is arranged on the surface region. Here a buffer substance is defined as a substance that produces a buffer solution when dissolved in a solvent.

A sensor chip can be easily manufactured from the blank obtained in this fashion, which initially has only the buffer solution without the receptor on its surface, in that a receptor with binding specificity for a ligand to be detected, which is dissolved in a solvent, is applied on the buffer substance in order to immobilize the receptor on the surface of the blank. Here the buffer substance causes the pH of the solution applied on the blank to be in a predefined range that is selected so that the receptor covalently binds to the reactive group of the support with reliability and with great effectiveness. The receptor solution that is to be applied to the sensor chip blank therefore does not itself need to contain any buffer substance; as a result of which, the risk of solids crystallizing, precipitating and/or separating out of the solution is avoided. The sensor chip blank according to the invention can be easily printed with the receptor using a matrix printer and/or a piezo stack printer without the risk of clogging the nozzle of the printer due to the formation of crystals. Here the application of at least one receptor can be done by the user of the sensor chip himself so that the user can easily produce different sensor chips adapted, in terms of the type and quantity of the receptors applied, to the measurement to be performed.

The surface region in which at least one reactive group of the support is arranged, is chemically inert; that is, it does not react with the buffer solution nor with the receptor to be applied thereon. The support can be comprised of a carrier with a surface layer containing bifunctional molecules applied thereon. Here the bifunctional molecules have a first reactive group that binds to the carrier and a second reactive group that binds covalently to the receptor. It is also possible, however, that the carrier itself comprises the reactive group and thus binds directly to the receptor.

The carrier may consist of a metal or semi-metal oxide such as silicon oxide, aluminum oxide, quartz glass or glass or it may have a surface layer comprised of this type of material. The carrier may also be comprised of a polymer material or it may have a surface layer made of this type of material. The polymer material may comprise a cyclo-olefin copolymer or a derivative thereof, polysterol or a derivative thereof, polyimide or a derivative thereof and/or polymethyl methacrylate or a derivative thereof. In addition, the carrier or a surface layer of the carrier may also have swellable or water-permeable polymeric or copolymer structures and include bi- or polyfunctional linking groups. Furthermore, the carrier may be comprised of a metal or semi-metal material or of a surface layer made of this type of material such as silicon, for example. The surface region in which at least one reactive group is arranged is preferably flat. It can also be uneven if, for example, structures for an electrical circuit are arranged under the surface region, particularly for an optical sensor for detecting luminescent radiation and/or an optical radiation source for transmitting an excitation radiation.

In an advantageous embodiment of the invention, at least two surface regions are provided on the support in which at least one buffer substance is arranged on each, wherein the buffer substances allocated to the individual surface regions differ from each other in their pH. Different types of receptors can be immobilized on the surface regions.

It is advantageous if at least one buffer substance contains at least one non-reactive dye. The buffer substance is then easily visible on the support and/or detectable with an optical sensor. The receptor can thus be very precisely brought into contact with the buffer substances. The dye can be methylene blue, for example.

Advantageously, the sensor chip blank has a machine-readable coding element for each of the different buffer substances. The coding element can, for example, include a color code and/or an electronic memory element in which a code allocated to the buffer substance can be saved. The code can then be read using an appropriate reader device in order to identify the individual buffer substances and then to apply to the buffer substance a receptor associated with the buffer substance in question.

In a preferred embodiment of the invention an indicator dye, whose color is pH-dependent, is arranged in at least one coating region. An analyte having a pH diverging from the pH of the buffer substance arranged in the coating region can then be applied to the coating region so that afterwards a pH is set in the analyte, which lies between the pH of the buffer substance and the pH the analyte had prior to being brought into contact with the buffer substance. The pH change produced in this fashion in the coating region can be visualized with the aid of the indicator dye. Thus it can be easily checked, whether the analyte has come into contact with the coating region and consequently has been deposited on the predefined site on the support. The pH of the analyte can be adjusted to a defined value before bringing the analyte in contact with the buffer substance, if required, by mixing the analyte with an acid or a base.

It is advantageous if a plurality of coating regions, arranged laterally separated from each other, preferably in a matrix in several rows and/or columns, are provided on the support. A micro-array can be easily produced using the sensor chip blank by the application of appropriate receptors.

In an advantageous embodiment of the invention the buffer substance is preferably a water-soluble salt, in particular sodium phosphate. These types of salt are available inexpensively and make possible high buffer capacities.

It is advantageous if the reactive group arranged in the surface region of the support is a chemoreactive group. By way of example, in the case of amino-terminated oligonucleotides, the so-called reactive esters such as N-hydroxysuccimide (NHS-ester), epoxide, and preferably the glycidyl derivatives, isothiocyanates, isocyanates, azides, carbonic acid groups or maleinimides, are suitable. Any number of techniques may be used for immobilization of the receptors such as those described by G. T. Hermanson in “Bioconjuncate Techniques” [sic]; Academic Press, 1996, for example.

In an advantageous embodiment of the invention the reactive group is a photoreactive group that is preferably capable of inducing free radicals when heat and/or optical radiation is applied. One such group is also designated as a photoreactive cross-linker. It may include at least one group selected from benzophenone or a derivative thereof, anthraquinone or a derivative thereof, thymidine or a derivative thereof and 4-azidobenzoic acid or a derivative thereof.

In a preferred embodiment of the invention, at least one coating area contains at least one tenside. By virtue of the tenside, receptors that contain folded molecules unfold after contact with the surface of the sensor chip blank. The molecules can thus be better immobilized on the blank. In addition, the tenside serves to keep low-solubility receptors in solution.

Preferably the tenside is chosen from the group comprising sodium palmitate, Brij® 35, Brij® 58, cetyl pyridinium chloride monohydrate, cetyl trimethyl ammonium bromide, 3-(3-cholamidopropyl) dimethylammonio-1-propane sulfonate, 3-(3-chloamidopropyl)-dimethylammonio-2-hydroxy-1-propane sulfonate, decane-1-sulfonic acid sodium salt, N,N-bis-[3-(D-gluconamido)-propyl]-deoxycholamide, dodecane-1-sulfonic acid sodium salt, dodecyl-β-D-maltoside, 6-0-(N-heptylcarbamoyl)-methyl-α-D-glucopyranoside, heptane-1-sufonic acid sodium salt, N-lauroylsarcosin sodium salt, octanoyl-N-methyl glucamide, N-nonanoyl-N-methyl glucamide, sodium cholate, sodium deoxycholate, nonane-1-sulfonic acid sodium salt, Nonidet P40, octane-1 sulfonic acid sodium salt, n-octyl-β-D-glucopyranoside, pentane-1-sulfonic acid sodium salt, n-octyl-β-D-thioglucopyranoside, Pluronic® F-68, saccharose monolaurate, sodium dodecyl sulfate, N-dodecyl-dimethyl-3-ammonio-1-propane sulfonate, N-tetradecyl-dimethyl-3-ammonio-1-propane sulfate, Triton® X-100 and/or mixtures thereof. In practice, good covalent binding of receptors to the surface of the support is observed with these tensides.

Further details of the aforementioned tensides are given in the following table:

| Identification | Chemical formula |
|---|---|
| Brij ® 35 | C₅₈H₁₁₈O₂₄ |
| Brij ® 58 | C₅₈H₁₁₄O₂₁ |
| cetyl pyridinium chloride monohydrate | C₂₁H₃₈CINxH₂₀ |
| cetyl trimethyl ammonium bromide | C₁₉H₄₂BrN |
| (3-(3-cholamidopropyl) | C₃₂H₅₈N₂O₇S |
| dimethylammonio-1-propanesulfonate) | |
| (3-(3-cholamidopropyl) | C₃₂H₅₈O₇N₂O₈S |
| dimethylammonio-2-propanesulfonate) | |
| dean-1-sulfonic acid sodium salt [sic] | C₁₀H₂₁NaO₃S |
| (N,N-bis-[3-(D-gluconamido]-propyl]-deoxychol- | C₄₂H₇₅N₃O₁₆ |
| amide) | |
| dodecane-1-sulfonic acid sodium salt | C₁₂H₃₅NaO₃S |
| dodecyl- 13-D-maltoside | C₁₂H₃₅NaO₃S [sic] |
| (6-O-(N-heptylcarbamoyl)-methyl-methyl-α-D- | C₁₅H₂₉NO₇ |
| glucopyranoside | |
| heptane-1-sufonic acid sodium salt | C₇H₁₅NaO₃S × H₂O |
| N-lauroyl sarcosin sodium salt | C₁₅H₂₈NNaO₃ |
| octanoyl-N-methyl glucamide | C₁₅H₃₁NO₆ |
| N-nonanoyl-N-methyl glucamide | C₁₆H₃₃NO₆ |
| sodium cholate | C₂₄H₃₉NaO₅ |
| sodium deoxycholate | C₂₄H₃₉NaO₄ |
| nonane-1-sulfonic acid sodium salt | C₉H₁₉NaO₃S |
| Nonidet P40 | mixture of 15 |
| | homologues |
| octane-i sulfonic acid sodium salt | C₈H₁₇NaO₃S |
| n-octyl-β-D-glucopyranoside | C₁₄H₂₈O₆ |
| pentane-1-sulfonic acid sodium salt | C₅H₁₁NaO₃S |
| n-octyl-β-D-thioglucopyranoside | C₁₄H₂₈O₅S |
| Pluronic ® F-68 | |
| saccharose monolaurate | C₂₄H₄₄O₁₂ |
| sodium dodecyl sulfate | C₂₄H₄₄O₁₂ [sic] |
| N-dodecyl-dimethyl-3-ammonio-1-propane sulfonate | C₁₇H₃₇NO₃S |
| N-tetradecyl-dimethyl-3-ammonio-1-propane sulfate | C₁₉H₄₁NO₃S |
| Triton ® X-100 | |
| Triton ® X-114 | |
| Tween ® 20 | |
| Tween ® 80 | |

A further aspect of the invention relates to a kit for use in the process according to the invention, which has the sensor chip according to the invention and at least one receptor solution that contains the at least one receptor dissolved in a solvent. Using this kit, sensor chips can be easily produced by application of the receptor solution to the sensor chip blank surface region containing the buffer substance. Here the receptor solution can be diluted, if necessary, prior to its application. Because the buffer substance comes into contact with the receptor solution only at the time of application of the receptor solution on the sensor chip blank, the kit can be stably stored over an extended period of time.

An exemplary embodiment of the invention will be more completely described below by means of the drawing, wherein:

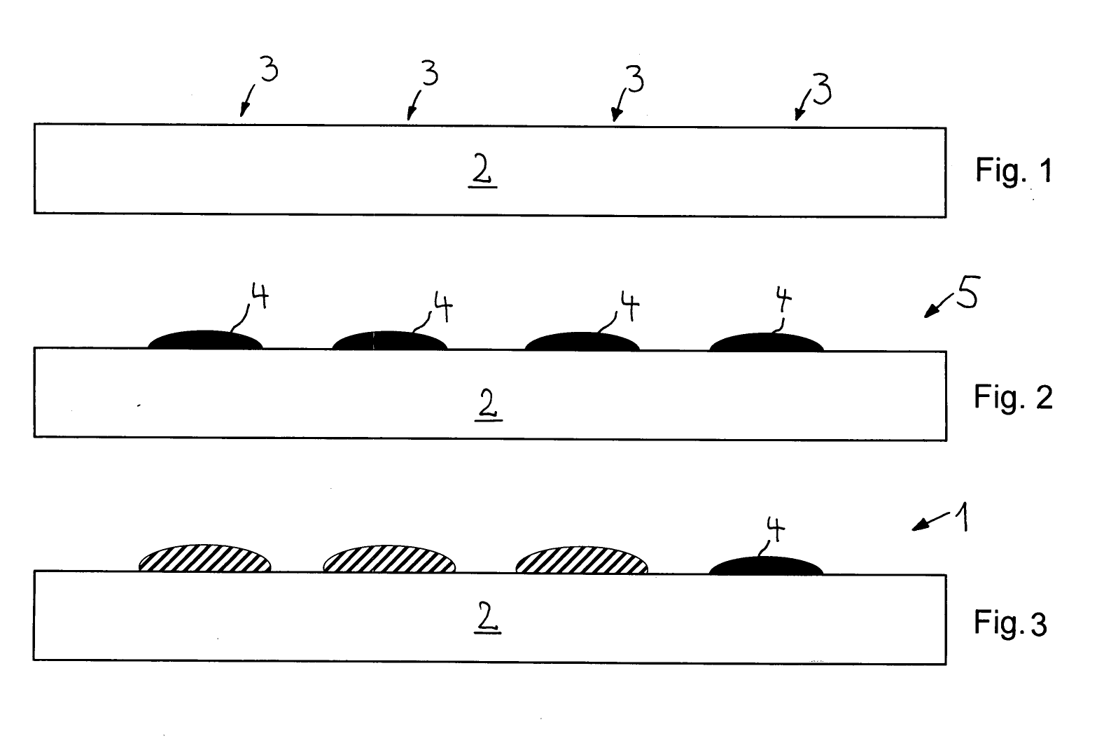

FIG. 1 represents a side view of a support for a sensor chip blank,

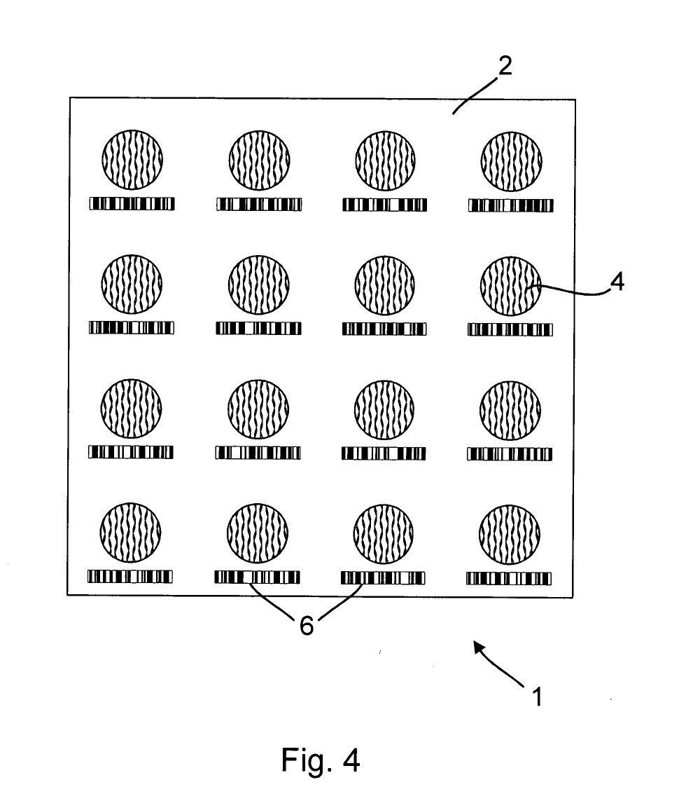

FIG. 2 represents a side view of a sensor chip blank, and

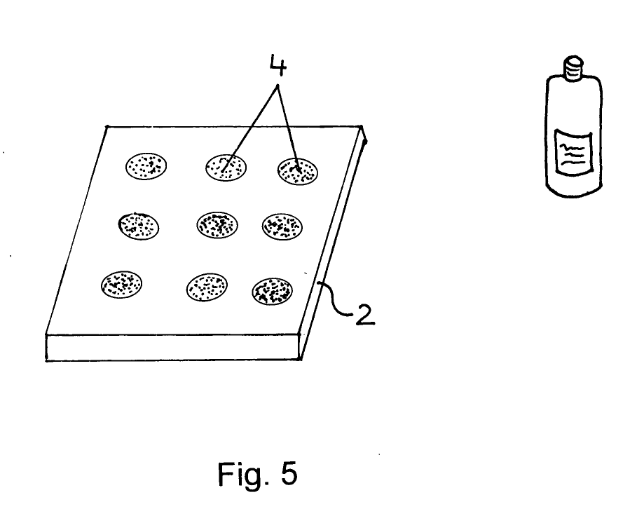

FIG. 3 represents a side view of a sensor chip.

In a process for manufacturing a biological sensor chip 1, a support 2 is prepared which has a surface region 3 in which reactive groups are arranged (not shown in greater detail in the drawing). The reactive groups can be amino or epoxy groups, for example.

The support 2 preferably has a solid carrier on which a thin surface layer is provided which contains the reactive groups. The surface layer is bonded to the support 2 and adheres to it.

Application of the reactive groups can be done using DIP coating in that the carrier is immersed into a solution that contains bifunctional molecules (so-called “linkers”), which the aforesaid reactive groups and a linking group on the surface of the carrier have. The last named group can be a halogen silane- (e.g. chlorsilane) or an alkoxysilane group.

In a further step, the carrier is withdrawn from the solution at a rate that is selected so that the molecules remain as a self-assembled monolayer (SAM) on the carrier after being withdrawn. The surface layer obtained in this fashion is covalently affixed to the carrier by cross-linking.

However, it is also conceivable that the material of the carrier itself contains the reactive groups. In this instance, the coating of the carrier with the surface layer can be omitted.

The carrier is comprised of a material that is inert to receptors that are applied to the support 2 in a process step described in more detail below. The carrier can, for example, be a glass plate, a polymer plate or a semi-conductor chip.

The support 2 is now coated with a buffer solution that contains a solvent and a buffer substance dissolved in it. The buffer solution can be a phosphate-buffered saline solution (PBS solution, 10-50 mM), a sodium phosphate buffer (100-500 mM), a sodium citrate chloride buffer (SSC buffer 1×-5×) or a betaine solution, for example. In addition, the buffer solution can contain a tenside in a concentration of 0.001-1% such as Triton® X-100 and/or Tween® 20, for example.

It can be seen in FIG. 2 that the support 2 is coated with the buffer solution at a plurality of matrix-like sites arranged separated from each other on its surface. The application of the buffer solution is done preferably using a pad printing process wherein a plurality of carrier 2 sites that are to be coated are coated simultaneously. The buffer solution can, however, also be applied to the carrier 2 by means of a matrix printer, a piezo stack printer or by other means.

The solvent that is contained in the solution situated on the carrier 2 is then evaporated so that the buffer substance and/or the tenside, if required, then remain on the individually coated sites as solid, paste-like and/or gel-like coated regions 4 on the carrier 2. The sensor chip blank 5 obtained in this fashion may be stored prior to use, if required, whereby the interim storage should be in a dry environment so that the coating area 4 does not absorb any moisture.

In a further process step, a solution containing the receptors is applied, preferably by means of a matrix or piezo stack printer, to the coating regions 4. The receptors are binding-specific for a specific ligand. After this solution has come into contact with the coating regions 4, the receptors contained in the solution bind covalently to the reactive groups present on the surface of the sensor chip blank 5. The solution is then removed by washing, for example, from the sensor chip blank 5 together with any remaining free receptors.

Using the sensor chip 1 obtained in this fashion, ligands can be detected in an analyte that is to be examined. The analyte is applied in liquid form to the coating regions 4 so that any ligands contained in the analyte can bind to the receptors immobilized on the support 2. Then the analyte is washed off the sensor ship 1 such that only the bound ligands remain on the sensor chip 1. As a factor of the binding of the ligands to the receptor a luminescence radiation is generated by conventional means and detected by means of an optical sensor. In the exemplary embodiment shown in FIG. 3 no receptors were immobilized on one of the coating regions 4 of the sensor chip blank 5. This coating region 4 can be used for reference or blind measurements.

1. A process for manufacturing a sensor chip, wherein a support is prepared that has at least one surface region in which at least one reactive group is arranged, wherein a buffer solution is applied to the surface in order to coat at least one surface region, and wherein at least one receptor with binding specificity for a ligand that is to be detected is brought into contact with the surface coating so obtained, such that the receptor binds covalently to at least one reactive group.

2. A process according to claim 1, characterized in that, after application of the buffer solution, a solvent contained in the buffer solution is removed from the surface region preferably by evaporation, so that a buffer substance contained in the buffer solution remains on the support as a solid, paste-like and/or gel-like coating region and so that the receptor is then applied to said coating region.

3. A process according to claim 1, characterized in that the buffer solution is applied to the at least one surface region of the support such that a plurality of buffer solution volumes are formed separated laterally, arranged preferably in a matrix in a plurality of rows and/or columns.

4. The process according to claim 1, characterized in that the buffer solution is applied by means of a printing process, in particular a pad printing process, to at least one surface region of the support.

5. The process according to claim 1, characterized in that at least one receptor is dissolved in a solvent and then printed or sprayed onto the surface region using a jet printer.

6. A sensor chip blank having a support that has at least one surface region in which at least one reactive group is arranged that is capable of covalently bonding to a receptor, characterized in that at least one solid, paste-like and/or gel-like coating region containing at least one buffer substance is arranged on the surface region.

7. A sensor chip blank according to claim 6, characterized in that at least two surface regions are provided on the support in each of which at least one buffer substance is arranged and that the buffer substances allocated to the individual surface regions differ from each other in their pH.

8. A sensor chip blank according to claim 6, characterized in that at least one buffer substance contains at least one non-reactive dye.

9. A sensor chip blank according to claim 6, characterized in that it has at least one machine-readable coding element for each of the different buffer substances.

10. A sensor chip blank according to claim 6, characterized in that an indicator dye is arranged in at least one coating region and the color of said dye is pH-dependent.

11. A sensor chip blank according to claim 6, characterized in that a plurality of coating regions are provided, arranged laterally separated from each other, preferably in a matrix in a plurality of rows and/or columns on the carrier.

12. A sensor chip blank according to claim 6, characterized in that the buffer substance is a preferably water-soluble salt, in particular sodium phosphate.

13. A sensor chip blank according to claim 6, characterized in that the reactive group is a chemoreactive group.

14. A sensor chip blank according to claim 6, characterized in that the reactive group is a photoreactive group that is preferably capable of inducing free radicals upon the application of heat and/or optical radiation.

15. A sensor chip blank according to claim 6, characterized in that at least one coating area contains at least one tenside.

16. A sensor chip blank according to claim 6, characterized in that the tenside is chosen from the group comprising sodium palmitate, Brij® 35, Brij® 58, cetyl pyridinium chloride monohydrate, cetyl trimethyl ammonium bromide, 3-(3-cholamidopropyl)-dimethylammonio-1-propane sulfonate, 3-(3-cholamidopropyl)-dimethylammonio-2-hydroxy-1-propane sulfonate, decane-1-sulfonic acid sodium salt, N,N-bis-[3-(D-gluconamido)-propyl]-deoxycholamide, dodecane-1-sulfonic acid sodium salt, dodecyl-β-D-maltoside, 6-0-(N-heptylcarbamoyl)-methyl-α-D-glucopyranoside, heptane-1-sufonic acid sodium salt, N-lauroylsarcosin sodium salt, octanoyl-N-methyl glucamide, N-nonanoyl-N-methyl glucamide, sodium cholate, sodium deoxycholate, nonane-1-sulfonic acid sodium salt, Nonidet P40, octane-1 sulfonic acid sodium salt, n-octyl-β-D-glucopyranoside, pentane-1-sulfonic acid sodium salt, n-octyl-β-D-thioglucopyranoside, Pluronic® F-68, saccharose monolaurate, sodium dodecyl sulfate, N-dodecyl-dimethyl-3-ammonio-1-propane sulfonate, N-tetradecyl-dimethyl-3-ammonio-1-propane sulfate, Triton® X-100 and/or mixtures thereof.

17. A kit for use in a process according to claim 1, characterized in that it has a sensor chip blank and at least one receptor solution, which contains at least one receptor dissolved in a solvent.

18. The kit according to claim 17, wherein said sensor chip blank has a support that has at least one surface region in which at least one reactive group is arranged that is capable of covalently bonding to a receptor, characterized in that at least one solid, paste-like and/or gel-like coating region containing at least one buffer substance is arranged on the surface region.
